(19) United States
(12) Patent Application Publication
(10) Pub. No.: US 2026/0263763 A1
(43) Pub. Date: Sep. 10, 2026

(54) H DRAIN FOR BILIARZ DRAINAGE

(71) Applicant: Emsad Halilovic, Sarajevo (BA)

(72) Inventor: Emsad Halilovic, Sarajevo (BA)

(21) Appl. No.: 19/166,605

(22) PCT Filed: Mar. 21, 2024
(86) PCT No.: PCT/BA2024/000001

§ 371 Date: Sep. 18, 2025

(30) Foreign Application Priority Data
Mar. 23, 2023 (BA) BAP233519A

(51) Int. Cl. A61M 27/00; A61M 25/00; A61M 25/10

(52) U.S. Cl. CPC A61M 27/002; A61M 25/007; A61M 25/10; A61M 2027/004; A61M 2202/0403; A61M 2210/106

(57) ABSTRACT

A H drain for biliary drainage to establish natural drainage of bile from the bile ducts into the small intestine. The H drain is an internal bile drain that plays a crucial role in the drainage of bile in conditions of malignant biliary obstruction in cases of pancreatic head carcinoma, Vater's papilla, duodenum, cholangiocarcinoma, and hepatocellular carcinoma when, due to locally advanced disease and metastases, they are unsuitable for curative surgery. The H drain has an important role in conditions of altered anatomy, obstruction of the gastric outlet, and previous gastric bypass surgery. The H drain provides the possibility of liver function recovery and nutritional status improvement, reduction of systemic endotoxemia, and enhancement of the immune response through internal drainage and restoration of bile flow into the intestinal lumen. The H drain can flush, unclog in case of obstruction, take bile samples, and contrast imaging of the bile ducts.

### FIELD OF THE INVENTION

This invention relates to a structural tube named the H drain for biliary drainage, whose purpose is to establish natural drainage of bile from the bile ducts into the small intestine, and is classified according to the international classification (IPC) as A61F; A61F 2/00.

### Technical Problem

The obstruction of bile ducts with the consequent appearance of jaundice is one of the frequent conditions due to which patients are hospitalized. Jaundice can occur due to biliary obstruction by cholangiocarcinoma, pancreas, duodenum, gall bladder, or primary or secondary liver malignancies. Despite the advent of multidisciplinary cancer therapy, pancreatic adenocarcinoma and peripancreatic cancer remain among the most morbid and deadliest carcinomas in the world. Bile is a liquid produced by the liver, consisting of bile salt, electrolytes, bilirubin (a product of hemoglobin breakdown), cholesterol, and other lipids. Malignant obstructive jaundice can occur at any level within the biliary tree, resulting in the bile ducts being unable to deliver bile to the duodenum, leading to a range of pathophysiological disorders in multiple organs and systems, such as electrolyte imbalance, weakened immunity, malnutrition, coagulation disorder, reduced digestion, and absorption. If bile drainage is not established within a short period, it can lead to infection of the biliary tract, liver and kidney failure, reduced quality of life, and increased mortality rate. The main symptoms and signs of bile duct obstruction are jaundice, itching, malabsorption, steatorrhea, and among the most serious complications is the occurrence of kernicterus, which represents brain damage caused by the deposition of bilirubin in the basal ganglia and brainstem nuclei.

### State of the Art

The only way to cure is through surgery, however, most patients present at a late stage, and due to locally advanced disease or metastasis, they are unsuitable for curative surgery, which is why palliative methods are most applied nowadays in such patients.

These methods aim at alleviating malignant obstructive jaundice, reducing bilirubin levels in the blood, protecting liver function, improving nutritional status, prolonging life, and thereby improving the quality of life. Endoscopic retrograde cholangiopancreatography (ERCP) and percutaneous transhepatic cholangial drainage (PTCD) are fundamental for palliative intervention. The installation of a metal stent using ERCP through the tumor leads to subsequent secondary stent dysfunction, ingrowth and/or excessive growth of the stent into the tumor tissue, then changed or variant anatomy, obstruction of the gastric outlet, permanent duodenal stent, and previous gastric bypass surgery are considered the most common reasons for ERCP failure. In such cases, percutaneous biliary drainage (PTC) or surgery is the standard treatment for biliary drainage. PTC involves the puncturing of the liver under fluoroscopy or ultrasound control to cannulate peripheral intrahepatic bile ducts above the common hepatic duct into which contrast is injected. Long-term therapy with the PTC procedure is associated with multiple complications such as cholangitis, dislocation of drains, and occlusion, leading to frequent reinterventions and hospital stays. Moreover, this leads to a poorer quality of life, as patients remain with long-term external drainage for the rest of their lives. Bile flows within the PTC with small pressure gradients. Most studies on external biliary drainage have not been able to show a reduction in complication rates in people because this procedure, while relieving biliary obstruction, does not restore the flow of bile into the lumen of the intestine, which is crucial for improving liver function and nutritional status, for reducing systemic endotoxemia, and for improving immune response. EUS-BD (Endoscopic Ultrasound-guided Biliary Drainage) is a new technique and is increasingly used as an alternative procedure to surgery or PTC for malignant biliary drainage in patients with failed ERCP due to its minimally invasive nature. EUS-BD has developed and is now a feasible alternative to PTC in high-volume centers. EUS-BD has certain advantages.

First, it is a less invasive and more physiological procedure for biliary drainage.

Furthermore, it avoids the need for long-term external body drainage, has a lower rate of reintervention, ensures better absorption of nutrients from the gastrointestinal tract, and avoids excessive loss of body electrolytes. However, EUS-BD is also associated with certain problems. This procedure is technically varied and complex and requires a high level of skill and specialized training to perform. Moreover, it is feasible only when performed by an experienced gastroenterologist in a large medical center with appropriate surgical and interventional radiological support for managing any acute complications such as bleeding, bile leakage, and pneumoperitoneum. Moreover, transmural puncture of the luminal side of the gastrointestinal tract to approach the sterile biliary tree may cause biliary infection.

### SUMMARY OF THE INVENTION

There are two types of H drains for biliary drainage. The difference between these two types lies in the vertical tube for the small intestine, where in the first type the vertical tube for the small intestine is in the shape of a T, while in the second type the tube is straight and can be with a balloon that prevents the drain from falling out or without a balloon where the tube is directly fixed to the intestine with stitches. The primary goal of the H drain for biliary drainage is to establish natural drainage of bile from the bile duct into the small intestine in conditions of advanced cancer when surgical resection and removal of the tumor causing obstruction of the bile ducts are not possible. The H drain provides the possibility of recovering liver function and nutritional status, reducing systemic endotoxemia, and improving immune response through internal drainage and restoring the flow of bile into the lumen of the intestine. It can be applied in both high and low unresectable lesions of the hepaticholedochus. It leaves the possibility of irrigation, unclogging in case of obstruction, taking bile samples for analysis, applying therapy, and contrast imaging of the bile ducts. It can also be used as a temporary method aimed at improving the overall status of the patient and preparing for surgical resection or for conducting palliative radio or chemotherapy. Given the manufacturing of the H drain and the method of its placement, postoperative complications are minimized. It significantly improves the quality of life of the patient, which is the basic goal of palliative care. It has a positive psychological impact on the patient, by eliminating the need for external drainage and the use of a bag for biliary content. It reduces the need for reinterventions.

It allows for a shorter hospitalization. Inexpensive procedure. And easy to apply and place, regardless of tumor stage, altered anatomical relationships and inaccessibility of the main bile duct.

### BRIEF DESCRIPTION OF THE DRAWINGS

The images that are included in the description and explanation of the invention are as follows:

FIG. 1. Appearance of the H drain for the first type of biliary drainage;

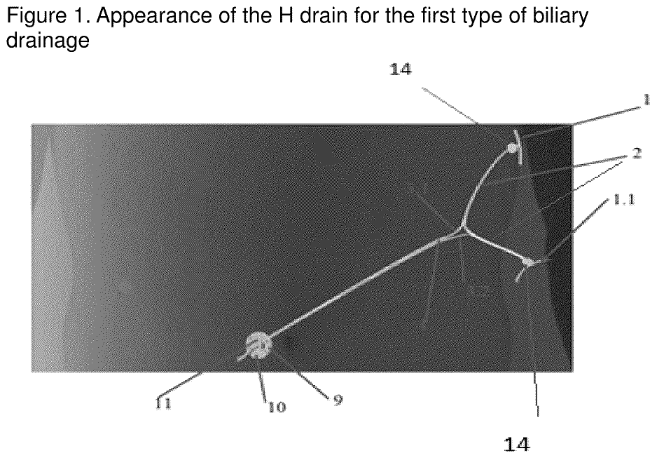

FIG. 2. Appearance of the internal part of the H drain for the first type of biliary drainage located in the abdominal cavity;

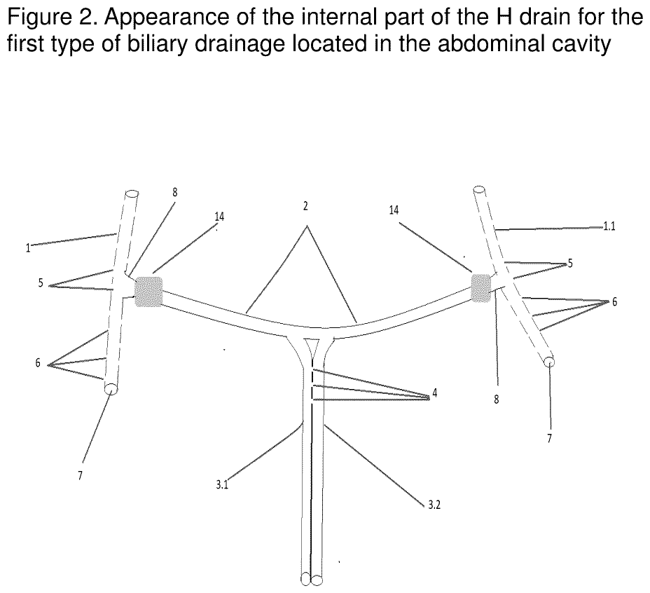

FIG. 3. Appearance of the vertical tube for the bile duct and small intestine for the H drain for the first type of biliary drainage and for the bile duct at the H drain for the second type of biliary drainage;

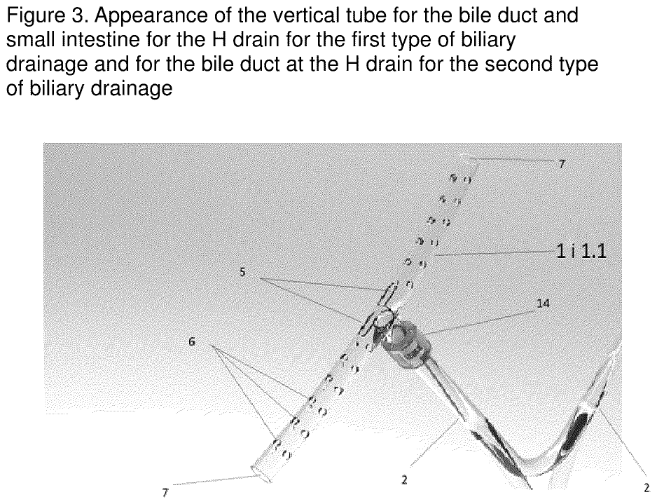

FIG. 4. Appearance of the H drain for the second type of biliary drainage without a balloon;

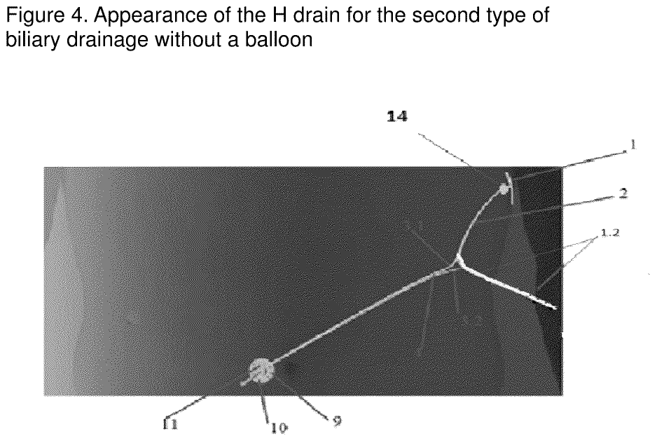

FIG. 5. Appearance of the vertical tube for the small intestine without a balloon for the H drain for the second type of biliary drainage;

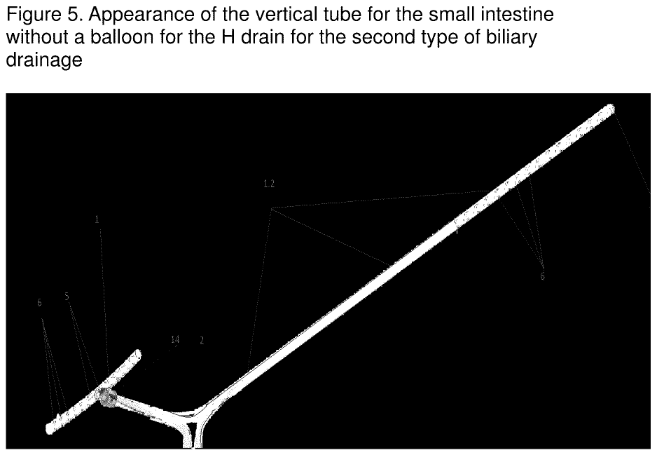

FIG. 6. Appearance of the vertical tube for the small intestine without a balloon for the H drain for the second type of biliary drainage;

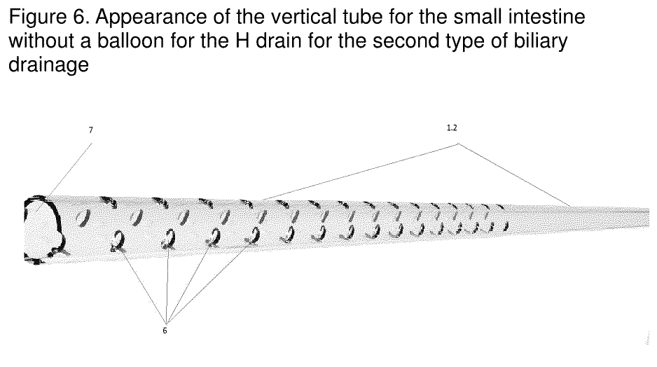

FIG. 7. Appearance of the H drain for the second type of biliary drainage with a balloon;

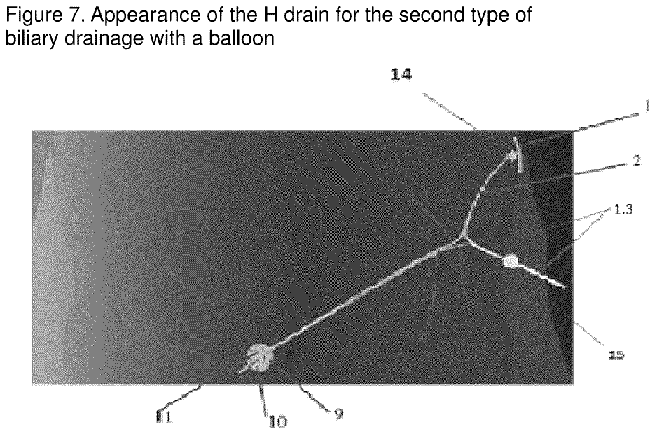

FIG. 8. Appearance of the H drain for the second type of biliary drainage with a balloon;

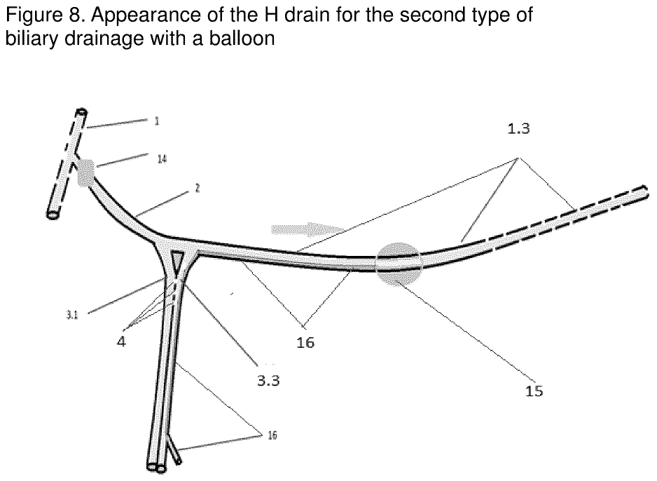

FIG. 9. Appearance of the vertical tube for the small intestine with a balloon for the H drain for the second type of biliary drainage;

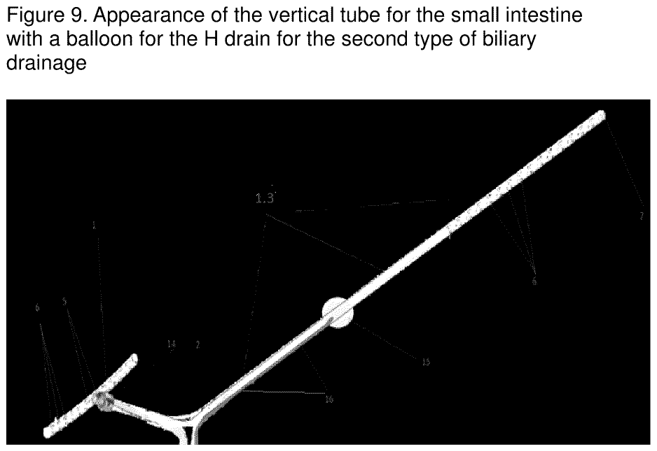

FIG. 10. Representation of the communication of the outlet tubes at the beginning part of the H drain for the first type of biliary drainage and the H drain for the second type of biliary drainage without a balloon;

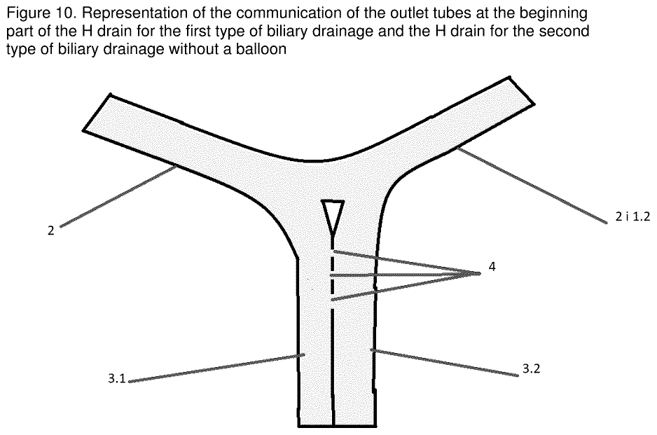

FIG. 11. Representation of the communication of the outlet tubes at the beginning part of the H drain for the second type of biliary drainage with a balloon;

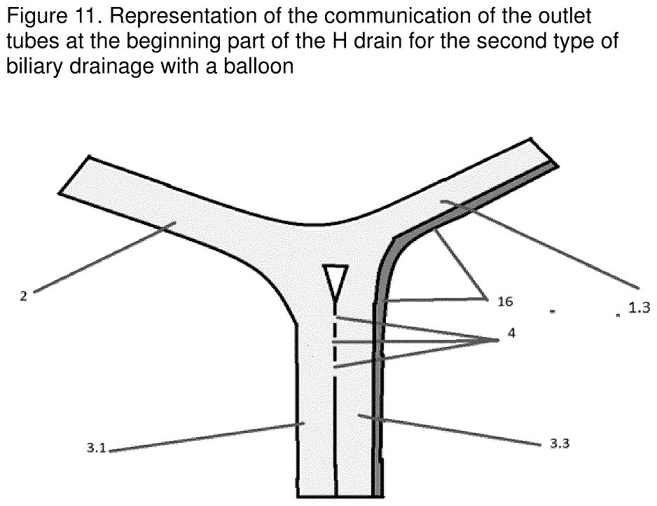

FIG. 12. Representation of the outlet tubes at the H drain for the first type of biliary drainage and the H drain for the second type of biliary drainage without a balloon;

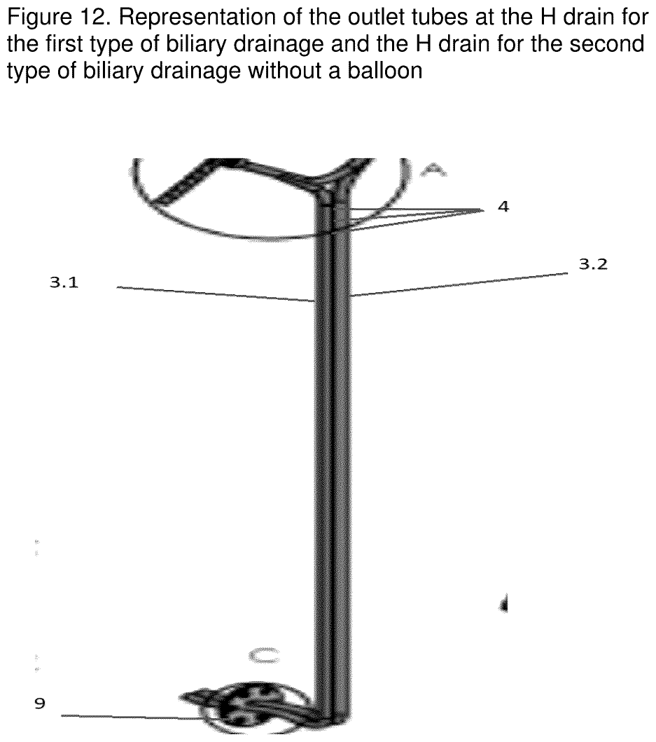

FIG. 13. Representation of the outlet tubes at the H drain for the second type of biliary drainage with a balloon;

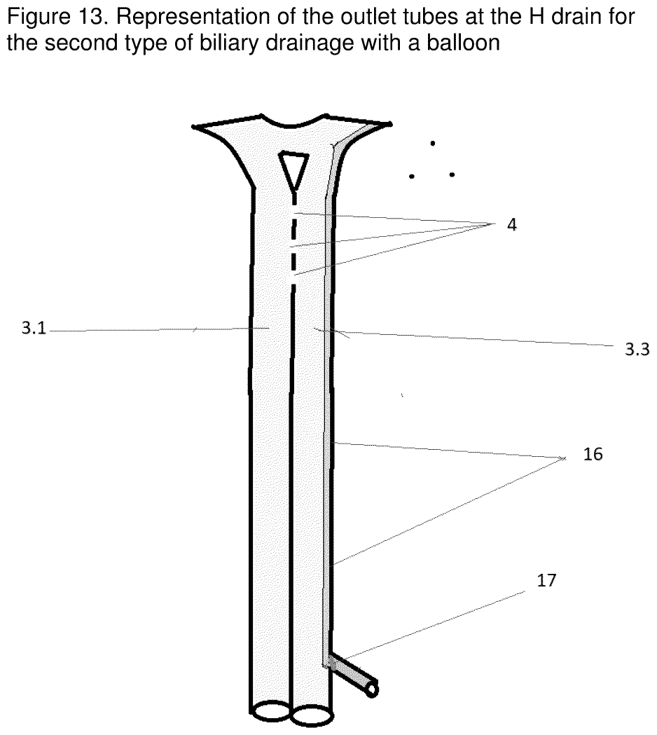

FIG. 14. Display of the silicone ring and clamps for fixation to the skin and securing the outlet tubes;

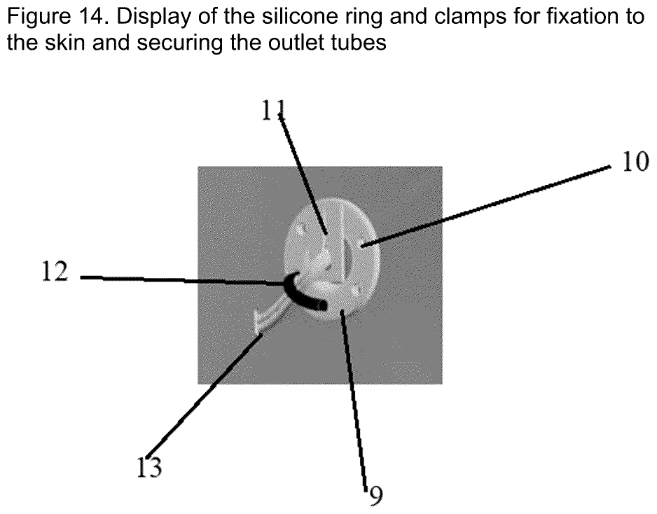

FIG. 15. Demonstration of the application of the H drain for the first type of biliary drainage;

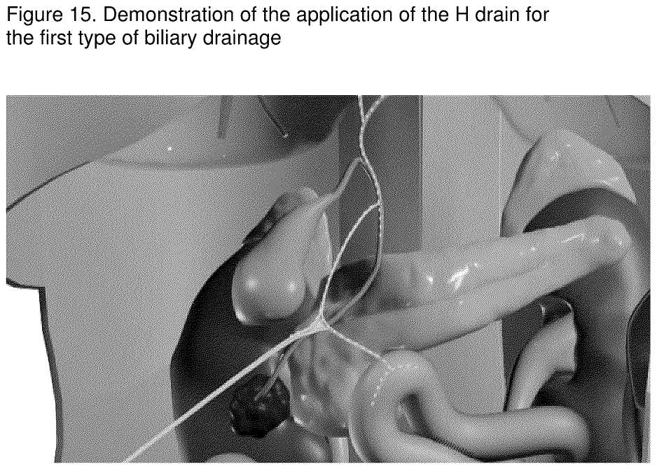

FIG. 16. Demonstration of the application of the H drain for the second type of biliary drainage without a balloon;

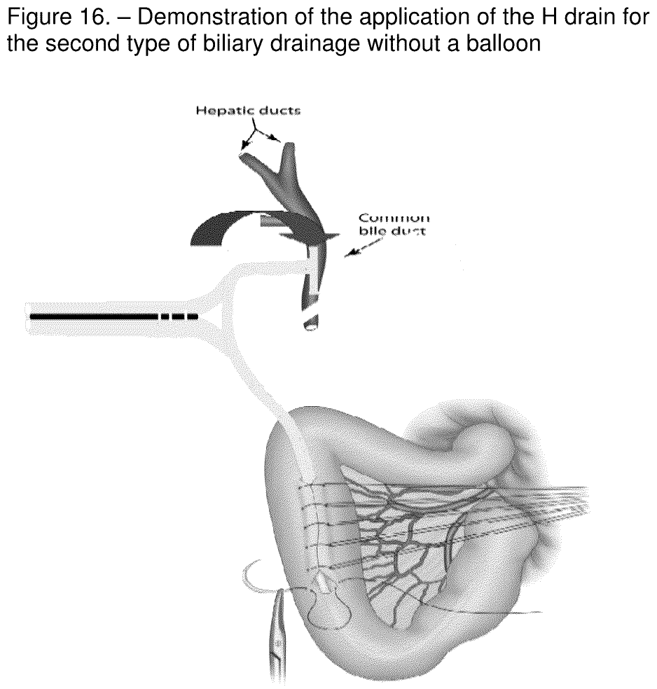

FIG. 17.-Demonstration of the application of the H drain for the second type of biliary drainage with a balloon; and

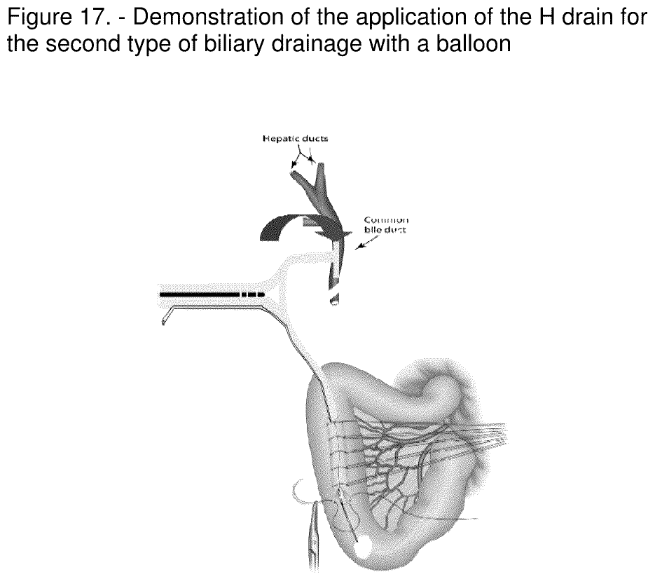

FIG. 18. Display of the H drain for biliary drainage with a valve in the vertical tube for the small intestine.

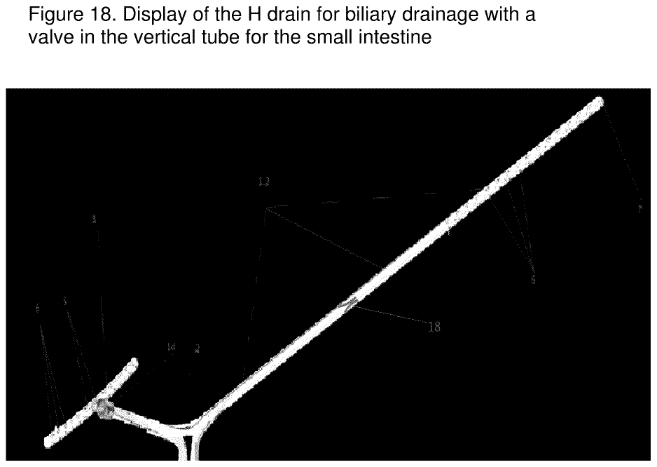

### DETAILED DESCRIPTION OF AT LEAST ONE MODE OF CARRYING OUT THE INVENTION

The H drain for biliary drainage represents a single unit consisting of two branches. One branch of the H drain is placed in the main bile duct, while the other branch is placed in the initial part of the small intestine. From the middle of the H drain, a double tube starts, which passes through the abdominal wall and is brought outside approximately about 5-6 cm; the extended part of the tube is fixed to the skin with a stitch, then it is passed through a silicone ring which is secured to the skin with a few individual stitches and acts as additional fixation for the blindly closed tube. The double tube that is brought outside has the role of irrigating the H drain, unclogging in case of obstruction, taking bile samples, and imaging the bile ducts—cholangiography. The H drain can be made of various materials such as silicone, latex, PVC, rubber, and others, coated with two biocidal substances, octenidine and polyhexanide, which have shown the best activity against two tested bacterial species E. coli and P. aeruginosa.These are also the most common bacteria present in the bile ducts and are the main causes of cholangitis. The H drain for biliary drainage comes in various diameters from 6 Fr to 40 Fr. The length of the vertical tube for the bile duct and small intestine, as well as the length of the drainage tube and outlet tubes, can be of various dimensions and diameters. The H drain can be placed using different surgical techniques and approaches.

### Method of Application of the Invention

The H drain is applicable for palliative treatment in patients with advanced carcinomas with biliary obstruction at all levels, contributing to longer survival, a lower percentage of complications, and improvement of the quality of life.

### LIST OF REFERENCE NUMERALS USED

1—Vertical tube placed in the bile duct
1.1—Vertical tube placed in the small intestine (first type)
1.2—Vertical tube placed in the small intestine (second type) without balloon (15)
1.3—Vertical tube placed in the small intestine (second type) with balloon (15), containing a lumen (16) used for filling the balloon (15) with saline solution
2—Drainage tube connecting vertical tubes (1 and 1.1, 1 and 1.2, 1 and 1.3) and outlet tubes (3.1 and 3.2, 3.1 and 3.3)
3.1—Outlet tube for cleaning the drainage tube (2) towards the bile duct (1). In the H drain for the first type of biliary drainage, it extends from the middle of the drainage tube and goes out through the abdominal wall. In the H drain for the second type of biliary drainage, it extends from the connection point of the drainage tube to the bile duct (2) and vertical tube for the small intestine without balloon (1.2) or with balloon (1.3)
3.2—Outlet tube used for cleaning, irrigating, and unclogging the drainage tube (2) towards the small intestine, vertical tube for the small intestine of the first type (1.1) and for cleaning the vertical tube for the small intestine of the second type without balloon (1.2). In the H drain for the first type of biliary drainage, it extends from the middle of the drainage tube (2) and goes out through the abdominal wall. In the H drain for the second type of biliary drainage, it extends from the connection point of the drainage tube to the bile duct (2) and vertical tube for the small intestine without balloon (1.2)
3.3—Outlet tube for cleaning the vertical tube for the small intestine of the second type with a balloon (1.3), containing a lumen (16) through which the balloon (15) is filled with saline solution. It extends from the connection point of the drainage tube to the bile duct (2) and the vertical tube for the small intestine of the second type with a balloon (1.3) and goes out through the abdominal wall
4—Perforated openings that allow communication between outlet tubes 3.1 and 3.2, 3.1 and 3.3 through which additional flow of biliary content occurs
5—Central openings on vertical tubes (1 and 1.1)
6—Perforated openings on vertical tubes (1, 1.1, 1.2, and 1.3)
7—Opening of tubes (1, 1.1, 1.2, 1.3, 3.1, 3.2, and 3.3) whose diameters can range from 6 Fr to 40 Fr
8—Coupling of vertical tube (1 and 1.1) with drainage (2) at a defined angle
9—External silicone ring for securing outlet tubes
10—Holes in the silicone ring for securing the ring to the skin
11—Plastic surgical clamp
12—Strap for securing outlet tubes (3.1, 3.2, and 3.3)
13—Cap for closing outlet tubes (3.1, 3.2, and 3.3)
14—Dacron cuff-cuff on the drainage tube (2) at the connection point with vertical tube (1 and 1.1)
15—Balloon located around the vertical tube for the small intestine of the second type (1.3), which is filled with saline solution through the lumen (16) located inside the outlet tube (3.3) and vertical tube for the small intestine of the second type (1.3), serving to retain the vertical tube (1.3) in the small intestine and prevent the drain from falling out
16—Lumen located inside the outlet tube (3.3) and vertical tube for the small intestine of the second type (1.3), ending in the balloon (15), through which the balloon is filled with saline solution from 1 ml to 10 ml.
17—Valve allowing the balloon (15) to be filled through the lumen located inside the outlet tube (3.3) and vertical tube for the small intestine with a balloon (1.3), and preventing fluid leakage from the balloon (15)
18—Valve inside the vertical tube for the small intestine (1.1, 1.2, and 1.3) allowing one-way.

1. A H drain system for biliary drainage comprising:
a first part adapted to be located inside an abdominal cavity, serving to conduct a bile content from bile ducts to small intestines, and
a second part adapted to be located outside on the skin for fixation, the second part serving for irrigation, unclogging of the drain, bile sampling, and contrast imaging of the bile ducts;
wherein in a first type system of the H drain system a vertical tube is adapted to be placed in the bile duct (1) is identical to a vertical tube adapted to be placed in the small intestine (1.1), the vertical tube placed in the bile duct (1) along with a drainage tube (2) and the vertical tube placed in the small intestine (1.1) forms an “H” shape; wherein the vertical tubes (1 and 1.1) in the central part opposite the junction with the drainage tube (2) have two larger perforated openings (5) from 0.1 mm to 20 mm in diameter, serving for the conduction and drainage of bile content, wherein the vertical tubes (1 and 1.1) contain a larger number of smaller perforated openings (6) which range from 1 to 100, with diameters from 0.1 mm to 10 mm, also serving for the conduction and drainage of bile content, wherein the vertical tubes (1 and 1.1) join with the drainage tube (2) at an obtuse angle in the form of a funnel (8) to enable smooth flow of bile without retention and obstruction, wherein at the junction of the vertical tube (1 and 1.1.) with the drainage tube (2) there is a dacron cuff (14) sleeve incluidng an antibacterial solution and allows the fixation of the drainage tube (2) to the bile duct and small intestine, wherein from the middle of the drainage tube (2), the outlet tubes (3.1 and 3.2) extend, going outside from the abdominal cavity through the abdominal wall and are fixed to the skin, wherein the outlet tubes (3.1 and 3.2) join the drainage tube (2) at a certain angle and then extend in their entirety outside through the abdominal wall, wherein the outlet tubes (3.1 and 3.2) communicate with each other at their starting section via perforated openings (4) which can range from 1 to 20, with diameters from 0.1 mm to 20 mm and serve for the additional flow of bile content and prevention of fluid retention in the tubes;
wherein ina a second type system of H drain system, the vertical tube for the bile duct (1) as well as the drainage tube to the bile duct (2) are identical to those of the H drain for biliary drainage the first type, wherein a difference between the first type system and second type system is in an appearance of the vertical tube for the small intestine, which for the second type system is straight and is without a balloon (1.2) or with a balloon (1.3), whererin in the second type system, the drainage tube to the bile duct (2) directly continues to the vertical tube for the small intestine (1.2 and 1.3), wherein the vertical tubes for the small intestine (1.2 and 1.3) in their final part on a length from 1 to 40 cm or longer, depending on the length of the vertical tube, have perforated openings (6) which are from 1 to 200, with diameters from 0.1 mm to 10 mm, also serving for the conduction and drainage of bile content, and a part of the tube that is entirely in the small intestine, while the rest of the tube without perforated openings (6) is outside the small intestine, wherein the vertical tube for the small intestine (1.3) includes a balloon (15) inside contains a lumen (16) used for filling the balloon (15) with a physiological solution in quantities from 1 ml to 10 ml, wherein the balloon (15) is located in a central part around the vertical tube (1.3), and is placed on any part of the vertical tube (1.3) which is without perforated openings (6), wherein the balloon (15) is placed in the small intestine and has the role of preventing the drain from falling out of the small intestine, wherein from the location where the drainage tube (2) and the vertical tubes (1.2 and 1.3) connect in the second type system, the outlet tubes (3.1 and 3.2, 3.1 and 3.3) originate, which are identical to vertical tubes of the first type system, except that in the outlet tube (3.3) there is a lumen (16) along its entire length inside, which serves to fill the balloon (15) with saline solution, wherein the Iumen (16) is found only in the second type system with a balloon. Wherein the lumen (16) starts inside the outlet tube (3.3), extends its entire length, and continues inside the vertical tube for the small intestine (1.3) ending in the balloon (15) where it serves the purpose of filling the balloon (15) with the saline solution, wherein in an initial part, the lumen (16) has a valve (17) that prevents the fluid from leaking from the balloon (15), wherein the vertical tubes (1, 1.1, 1.2, and 1.3), the drainage tube (2), and the outlet tubes (3.1, 3.2, and 3.3) are open and fully passable (7), and are made from different materials such as silicone, latex, PVC, polyurethane, rubber
wherein the external part of the first type system and the second type system include a silicone ring (9) through which the outlet tubes (3.1, 3.2, and 3.3) are threaded wherein there are holes (10) on the silicone ring for fixing the ring to the skin, wherein a Plastic surgical clamp (11) is placed with the purpose of closing the outlet tubes and preventing the spillage of bile, wherein a Strap for fixation (12) of the outlet tubes (3.1 and 3.2, and 3.3) is located on the silicone ring and has an additional role in fixing the tubes,
wherein the outlet tubes (3.1, 3.2, and 3.3) are closed with a cap (13),
wherein the first type system and the second type system contain within the lumen of the vertical tubes for the small intestine (1.1, 1.2, and 1.3) a valve (18) adjusted to the diameter of the tube, size from 0.1 to 50 mm, which allows a one-way flow of fluid from the bile duct to the small intestine and thereby prevents the reflux of intestinal content from the small intestine into the bile duct,
wherein the outlet tubes (3.1, 3.2, and 3.3) are marked in millimeters from their starting point outward, to accurately gauge the distance of the drainage tube (2).

2. The H drain system according to claim 1, wherein the vertical tubes (1 and 1.1) have a length from 1 cm to 20 cm, with a diameter from 6 Fr to 40 Fr, with hollow ends that are passable (7), allowing the unobstructed flow of bile.

3. The H drain system according to claim 1, wherein the vertical tubes (1 and 1.1) have perforated holes (6) which range from 1 to 100, with diameters from 0.1 mm to 10 mm, wherein in the central part of the vertical tubes (1 and 1.1) opposite to the junction with the drainage tube (2) includes two larger perforated holes (5) with a diameter from 0.1 mm to 20 mm.

4. The H drain system according to claim 1, wherein the drainage tube (2) in the H drain for biliary drainage first type extends from the vertical tube for the bile duct (1) to the vertical tube for the small intestine (1.1) and has a length from 1 cm to 50 cm, with a diameter from 6 Fr to 40 Fr, wherein the tube is hollow passable at both of its ends where it connects with the vertical tubes (1 and 1.1), wherein-the drainage tube (2) connects at an obtuse angle (8) with the vertical tubes (1 and 1.1) with the aim of ensuring the unobstructed flow of bile content.

5. The H drain system for biliary drainage according to claim 1, wherein the drainage tube (2) at the junction with the vertical tubes (1 and 1.1) has a dacron cuff (14) that serves an antibacterial role and allows the fixation of the drainage tube (2) to the bile duct and small intestine, wherein the dacron cuff (14) is placed around the tube, encompasses the entire circumference of the tube, and has a size from 0.1 mm to 50 mm.

6. The H drain system according to claim 1, wherein the vertical tubes for the small intestine (1.2 and 1.3) in the H drain for biliary drainage second type are straight, and have a length from 1 cm to 70 cm, with a diameter from 6 Fr to 40 Fr.

7. The H drain system according to claim 1, wherein the vertical tubes for the small intestine in the H drain for biliary drainage second type (1.2 and 1.3) in a final part have a length from 1 to 30 cm, have perforated openings (6) from 1 to 200, with diameters from 0.1 mm to 20 mm, wherein a portion of the vertical tubes (1.2 and 1.3), final few centimeters, contains perforated openings (6) and a part of the tube is adapted to be placed in the small intestine, while the remainder of the tube towards the junction with the drainage tube (2) is intact without perforated openings and is adapted to be located outside the small intestine.

8. The H drain according to claim 1, wherein the vertical tube for the small intestine (1.3) contains a balloon (15) which is located in any part of the vertical tube (1.3) which has no perforated openings, wherein balloon (15) is filled with a saline solution from 1 ml to 10 ml, through the lumen (16) located inside the vertical tube for the small intestine (1.3) and the outlet tube (3.3).

9. The H drain system according to claim 1, wherein the drainage tube (2) directly continues into the vertical tube for the small intestine (1.2 and 1.3) and has a length from 1 cm to 50 cm, with a diameter from 6 Fr to 40 Fr. Wherein the drainage tube (2) is completely hollow and allows the unobstructed flow of bile content.

10. The H drain system according to claim 1, wherein the outlet tubes (3.1 and 3.2, 3.1 and 3.3) diverge at a specific angle from the drainage tube (2), after which they join and are assembled into one unit with separate drainage tubes that are brought out of the abdomen through the abdominal wall, wherein the outlet tubes within the abdominal cavity at the beginning part communicate with each other via perforated holes (4) which range from 1 to 20, with diameters from 0.1 mm to 20 mm, and serve the role of additional flow of bile content and preventing the retention of fluid within the tubes, wherein, the outlet tubes (3.1, 3.2, and 3.3) have a lengths from 5 cm to 80 cm, with diameters from 6 Fr to 40 Fr.

11. The H drain system according to claim 1, wherein the outlet tube (3.3) internally contains a lumen (16) for filling the balloon (15) with a saline solution, which is located on the vertical tube for the small intestine (1.3), where a length of the lumen (16) is adjusted to the length of the outlet tube (3.3) and contains a valve (17) in an initial part, which prevents the leakage of fluid from the balloon (15), wherein from the outlet tube (3.3), the lumen (16) continues further inside the vertical tube (1.3) and ends in the balloon (15), the Lumen (16) has a diameter from 2 Fr to 20 Fr depending on the diameter of the drain.

12. The H drain system according to claim 1, wherein the internal tubes (3.1, 3.2, and 3.3), vertical tubes (1, 1.1, 1.2, and 1.3), and the drainage tube (2) are coated with two biocidal substances, octenidine and polyhexanide, to prevent the development of bile duct inflammation caused by the bacteria E. coli and P. Aeruginosa.

13. The H drain system according to claim 1, wherein a silicone ring (9) which is placed on the abdomen has from 2 to 10 holes (10) that serve to fix the ring to the skin, wherein the outlet tubes (3.1, 3.2, and 3.3) are threaded through the silicone ring (9) and are then fixed to the skin with stitches.

14. The H drain system according to claim 1, wherein the plastic surgical clamp (11) clamps the outlet tubes (3.1, 3.2, and 3.3), thereby obstructing the outlet tubes (3.1, 3.2, and 3.3) in the external part and thus preventing the discharge of bile.

15. The H drain system according to claim 1, wherein additional fixation of the outlet tubes (3.1, 3.2, and 3.3) and achieving a natural appearance of the body is accomplished by connecting the outlet tubes with a strap (12) that is attached to a silicone ring (9).

16. The H drain system according to claim 1, wherein the outlet tubes (3.1, 3.2, and 3.3) for the complete obstruction of the discharge of bile content are closed at their top with a cap (13).

17. The H drain system according to claim 1, wherein the H drain of the first and second type contain within the lumen of the vertical tubes for the small intestine (1.1, 1.2, and 1.3) a valve (18) adapted to the diameter of the tube, sizes from 0.1 to 50 mm, which allows the unidirectional flow of fluid from the bile duct towards the small intestine and thus prevents the reflux of intestinal content from the small intestine into the bile duct, wherein the Valve (18) in the vertical tubes (1.2 and 1.3) is located in the part of the tube without perforated openings at any distance.

18. The H drain system according to claim 1, wherein the outlet tubes (3.1, 3.2, and 3.3) are marked in millimeters or another metric system for length, from their starting point outward, to gauge the distance at which the drainage tube (2) is located.
